(19) United States
(12) Patent Application Publication
(10) Pub. No.: US 2020/0368190 A1
(43) Pub. Date: Nov. 26, 2020

(54) METHODS OF TREATMENT

(71) Applicant: Arena Pharmaceuticals, Inc., San Diego, CA (US)

(72) Inventors: John W. ADAMS, San Diego, CA (US); Christen M. ANDERSON, Tulsa, OK (US); William R. SHANAHAN, Del Mar, CA (US)

(21) Appl. No.: 16/636,608

(22) PCT Filed: Aug. 7, 2018
(86) PCT No.: PCT/US2018/045683

§ 371 Date: Feb. 4, 2020

Related U.S. Application Data
(60) Provisional application No. 62/711,369, filed on Jul. 27, 2018.
(60) Provisional application No. 62/541,956, filed on Aug. 7, 2017.

(51) Int. Cl. A61K 31/195; A61P 43/00

(52) U.S. Cl. CPC A61K 31/195; A61K 9/0053; A61P 43/00

(57) ABSTRACT

Provided in some embodiments are methods of treating or preventing Raynaud's, comprising prescribing and/or administering to an individual in need thereof 2-(((1r,4r)-4-(((4-chlorophenyl)(phenyl)carbamoyloxy)methyl)cyclohexyl)methoxy)acetic acid (Compound 1), or a pharmaceutically acceptable salt, hydrate, or solvate thereof.

### FIELD OF THE INVENTION

Provided are methods useful in the treatment of Raynaud's. Raynaud's (also referred to as Raynaud/Raynaud's syndrome, Raynaud/Raynaud's disease, and/or Raynaud/Raynaud's phenomenon) is a condition in which vasospasm of the arteries reduces blood flow. During an “attack,” the vessels narrow and limit blood circulation to affected areas, typically involving the fingers and occasionally the toes, nose, or ears. Raynaud's is triggered by the sympathetic nervous system, cold exposure, and/or emotional stress. The most severe complication is critical digital ischemia that may lead to digital ulceration and occasionally gangrene.

Raynaud's presents in primary and secondary forms. The cause of primary Raynaud's is not known. Vascular abnormality with this form is thought to be purely functional and reversible without progressing to irreversible tissue injury. However, some cases of primary Raynaud's progress to secondary Raynaud's.

Secondary Raynaud's occurs as a result of another condition, such as systemic sclerosis (also referred to as systemic scleroderma), rheumatoid arthritis, atherosclerosis, cryoglobulinemia, hypothyroidism, injury, and/or drug reaction. For example, over 95% of individuals with systemic sclerosis also present with Raynaud's. The severity of secondary Raynaud's is related to the occurrence of structural and functional vascular abnormalities.

Raynaud's is complex and likely involves an interplay between vascular factors, neural control mechanisms, and intravascular factors. The treatment approach for Raynaud's is consistent in mild cases, but diverges in cases involving moderate to severe digital ulceration or underlying systemic sclerosis. Intravenous prostanoids (prostacyclins and analogues) are used in individuals who develop ischaemic digital complications. Intravenous iloprost has been shown to reduce weekly Raynaud phenomenon attacks (39.1% vs. 22.2%) and improve severity score (34.8% vs. 19.7%) over 9 weeks. Wigley 1994 Ann Intern Med. 1994 Feb. 1; 120(3):199-206 Intravenous epoprostenol has been associated with reduced frequency and duration of Raynaud phenomenon attacks, but loss in clinical response was observed 8-10 weeks after the last infusion. Belch 1983 Lancet. 1 (8320):313e5.

Even if intravenous administration were efficacious, it is inconvenient and not feasible for long-term treatment. Further, oral prostanoids are often poorly tolerated and offer little or no significant benefit. In a recent survey, only 16% of individuals found current medication for Raynaud's to be effective. Hughes et al. Rheum 2015. 54 (8): 1443-1447.

New compounds for the treatment of Raynaud's, particularly in oral form, are needed.

### SUMMARY OF THE INVENTION

Provided herein is a method of treating or preventing Raynaud's, comprising:

prescribing and/or administering to an individual in need thereof 2-(((1r,4r)-4-(((4-chlorophenyl)(phenyl)carbamoyloxy)methyl)cyclohexyl)methoxy)acetic acid (Compound 1) or a pharmaceutically acceptable salt, hydrate, or solvate thereof.

Also provided herein is the use of 2-(((1r,4r)-4-(((4-chlorophenyl)(phenyl)carbamoyloxy) methyl)cyclohexyl)methoxy)acetic acid (Compound 1) or a pharmaceutically acceptable salt, hydrate, or solvate thereof in the manufacture of a medicament for the treatment of Raynaud's in an individual in need thereof.

Also provided herein is 2-(((1r,4r)-4-(((4-chlorophenyl)(phenyl)carbamoyloxy)methyl) cyclohexyl)methoxy)acetic acid (Compound 1) or a pharmaceutically acceptable salt, hydrate, or solvate thereof for use in a method of treatment of Raynaud's in an individual in need thereof.

Also provided herein is 2-(((1r,4r)-4-(((4-chlorophenyl)(phenyl)carbamoyloxy)methyl) cyclohexyl)methoxy)acetic acid (Compound 1) or a pharmaceutically acceptable salt, hydrate, or solvate thereof in the treatment of Raynaud's in an individual in need thereof.

In some embodiments, a method of treating or preventing Raynaud's is a method of increasing digital blood flow.

In some embodiments, a method of treating or preventing Raynaud's is a method of improving digital blood pressure.

In some embodiments, a method of treating or preventing Raynaud's is a method of improving digital temperature.

In some embodiments, a method of treating or preventing Raynaud's is a method of increasing digital temperature.

In some embodiments, a method of treating or preventing Raynaud's is a method of increasing skin temperature.

In some embodiments, a method of treating or preventing Raynaud's is a method of increasing capillary diameter.

In some embodiments, a method of treating or preventing Raynaud's is a method of lessening capillary dysfunction.

In some embodiments, a method of treating or preventing Raynaud's is a method of decreasing digital capillary dysfunction.

In some embodiments, a method of treating or preventing Raynaud's is a method of improving microvascular reactivity.

In some embodiments, a method of treating or preventing Raynaud's is a method of improving microvascular flow.

In some embodiments, a method of treating or preventing Raynaud's is a method of decreasing the frequency, duration and/or severity of Raynaud's attacks.

In some embodiments, a method of treating or preventing Raynaud's is a method of decreasing the frequency, duration and/or severity of vasospastic episodes.

In some embodiments, a method of treating or preventing Raynaud's is a method of decreasing the frequency, duration and/or severity of vasospastic attacks in individuals with secondary Raynaud's.

In some embodiments, a method of treating or preventing Raynaud's is a method of decreasing the frequency, duration and/or severity of episodes of pallor.

In some embodiments, a method of treating or preventing Raynaud's is a method of decreasing the frequency, duration and/or severity of episodes of cyanosis.

In some embodiments, a method of treating or preventing Raynaud's is a method of decreasing the frequency, duration and/or severity of episodes of pain, tingling and/or numbness in the fingers and/or hands.

In some embodiments, a method of treating or preventing Raynaud's is a method of decreasing the frequency of vasospastic attacks.

In some embodiments, a method of treating or preventing Raynaud's is a method of decreasing the duration of vasospastic attacks.

In some embodiments, a method of treating or preventing Raynaud's is a method of decreasing the severity of vasospastic attacks.

In some embodiments, a method of treating or preventing Raynaud's is a method of decreasing the frequency of Raynaud's phenomenon attacks.

In some embodiments, a method of treating or preventing Raynaud's is a method of decreasing the duration of Raynaud's phenomenon attacks.

In some embodiments, a method of treating or preventing Raynaud's is a method of decreasing the severity of Raynaud's phenomenon attacks.

In some embodiments, a method of treating or preventing Raynaud's is a method of reducing the number of Raynaud's phenomenon attacks per day.

In some embodiments, a method of treating or preventing Raynaud's is a method of reducing the number of Raynaud's phenomenon attacks per week.

In some embodiments, a method of treating or preventing Raynaud's is a method of reducing the number of Raynaud's phenomenon attacks per month.

In some embodiments, a method of treating or preventing Raynaud's is a method of improving the symptoms and/or signs of primary Raynaud's phenomenon.

In some embodiments, a method of treating or preventing Raynaud's is a method of improving the symptoms and/or signs of secondary Raynaud's phenomenon.

In some embodiments, a method of treating or preventing Raynaud's is a method of preventing cold-induced vasospasm.

In some embodiments, a method of treating or preventing Raynaud's is a method of decreasing a Raynaud's Condition Score (RSC).

In some embodiments, a method of treating or preventing Raynaud's is a method of reducing ulcer burden.

In some embodiments, a method of treating or preventing Raynaud's is a method of reducing the time to heal active ulcers and/or reducing the number of new ulcers.

In some embodiments, Compound 1 is 2-(((1r,4r)-4-(((4-chlorophenyl)(phenyl) carbamoyloxy)methyl)cyclohexyl)methoxy)acetic acid or a pharmaceutically acceptable salt, hydrate, or solvate thereof. In some embodiments, Compound 1 is sodium 2-(((1r,4r)-4-(((4-chlorophenyl) (phenyl)carbamoyloxy)methyl)cyclohexyl)methoxy)acetate hydrate. In some embodiments, Compound 1 is 2-(((1r,4r)-4-(((4-chlorophenyl)(phenyl)carbamoyloxy)methyl)cyclohexyl)methoxy)acetic acid or a pharmaceutically acceptable salt thereof. In some embodiments, Compound 1 is sodium 2-(((1r,4r)-4-(((4-chlorophenyl)(phenyl)carbamoyloxy)methyl)cyclohexyl)methoxy)acetate. In some embodiments, Compound 1 is 2-(((1r,4r)-4-(((4-chlorophenyl)(phenyl)carbamoyloxy)methyl)cyclohexyl)methoxy) acetic acid or a hydrate or solvate thereof. In some embodiments, Compound 1 is 2-(((1r,4r)-4-(((4-chlorophenyl)(phenyl)carbamoyloxy)methyl)cyclohexyl)methoxy)acetic acid or a hydrate thereof. In some embodiments, Compound 1 is 2-(((1r,4r)-4-(((4-chlorophenyl)(phenyl)carbamoyloxy)methyl) cyclohexyl)methoxy)acetic acid or a solvate thereof. In some embodiments, Compound 1 is 2-(((1r,4r)-4-(((4-chlorophenyl)(phenyl)carbamoyloxy)methyl)cyclohexyl)methoxy)acetic acid. In some embodiments, Compound 1 is an anhydrous, non-solvated crystalline form of 2-(((1r,4r)-4-(((4-chlorophenyl)(phenyl)carbamoyloxy)methyl)cyclohexyl)methoxy)acetic acid.

In some embodiments, the individual has primary Raynaud's.

In some embodiments, the individual has secondary Raynaud's.

In some embodiments, the individual has at least one digital ulcer.

In some embodiments, the individual has digital gangrene.

In some embodiments, the individual has systemic sclerosis.

In some embodiments, the individual has Raynaud's secondary to a condition selected from: systemic sclerosis, rheumatoid arthritis, atherosclerosis, cryoglobulinemia, hypothyroidism, injury, and drug reaction.

In some embodiments, the individual has Raynaud's secondary to systemic sclerosis.

In some embodiments, the individual has at least one digital ulcer and systemic sclerosis.

In some embodiments, the individual has a history of digital infarcts. In some embodiments, the individual has a history of digital ulcerations. In some embodiments, the individual has impending potential for digital amputation. In some embodiments, an individual has 1 to 3 fingers that are symptomatic for Raynaud's Phenomenon under a standard of care treatment. In some embodiments, an individual has 1 finger that are symptomatic for Raynaud's Phenomenon under a standard of care treatment. In some embodiments, an individual has 2 fingers that are symptomatic for Raynaud's Phenomenon under a standard of care treatment. In some embodiments, an individual has 3 fingers that are symptomatic for Raynaud's Phenomenon under a standard of care treatment. In some embodiments, an individual has more than 3 fingers that are symptomatic for Raynaud's Phenomenon under a standard of care treatment.

In some embodiments, the individual is unresponsive to a standard therapy. In some embodiments, the individual is resistant to vasodilatory therapy.

In some embodiments, the individual is administered an amount equivalent to 0.01 mg to 1.5 mg of Compound 1 daily.

In some embodiments, the individual is administered an amount equivalent to 0.01 mg to 1.0 mg of Compound 1 daily.

In some embodiments, the individual is administered an amount equivalent to up to 0.6 mg of Compound 1 daily.

In some embodiments, the individual is administered an amount equivalent to up to 0.8 mg of Compound 1 daily.

In some embodiments, the dose of the Compound 1 or pharmaceutically acceptable salt, hydrate, or solvate thereof is selected from: 0.01 mg, 0.02 mg, 0.025 mg, 0.03 mg, 0.04 mg, 0.05 mg, 0.06 mg, 0.065 mg, 0.07 mg, 0.075 mg, 0.08 mg, 0.09 mg, 0.1 mg, 0.12 mg, 0.15 mg, 0.16 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, and 1.5 mg daily.

In some embodiments, the dose is administered once daily.

In some embodiments, the dose is administered twice daily.

In some embodiments, Compound 1 is administered before cold exposure to the individual. For example, in some embodiments, Compound 1 is administered one day before cold exposure to the individual. In some embodiments, Compound 1 is administered immediately preceding cold exposure to the individual. In some embodiments, Compound 1 is administered during cold exposure to the individual. In some embodiments, Compound 1 is administered following the appearance of symptoms during cold exposure to the individual.

In some embodiments, the Compound 1 or pharmaceutically acceptable salt, hydrate, or solvate thereof is suitable for administration once daily.

In some embodiments, the Compound 1 or pharmaceutically acceptable salt, hydrate, or solvate thereof is suitable for administration twice daily.

In some embodiments, the Compound 1 or pharmaceutically acceptable salt, hydrate, or solvate thereof is administered via a titration scheme that comprises up-titration of the Compound 1 or pharmaceutically acceptable salt, hydrate, or solvate thereof.

In some embodiments, the titration scheme comprises increasing the dose, wherein the cycle is repeated so long as the individual tolerates a further increased dose, until an optimized dose is administered.

In some embodiments, the optimized dose is selected from: 0.01 mg, 0.02 mg, 0.025 mg, 0.03 mg, 0.04 mg, 0.05 mg, 0.06 mg, 0.065 mg, 0.07 mg, 0.075 mg, 0.08 mg, 0.09 mg, 0.1 mg, 0.12 mg, 0.15 mg, 0.16 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, and 1.5 mg daily.

In some embodiments, the optimized dose is administered once daily.

In some embodiments, the optimized dose is administered twice daily.

In some embodiments, the Compound 1 or pharmaceutically acceptable salt, hydrate, or solvate thereof is administered orally.

In some embodiments, the Compound 1 or pharmaceutically acceptable salt, hydrate, or solvate thereof is in the form of a tablet.

In some embodiments, the Compound 1 or pharmaceutically acceptable salt, hydrate, or solvate thereof is in the form of a capsule.

In some embodiments, the Compound 1, or a pharmaceutically acceptable salt, hydrate, or solvate thereof, is Compound 1.

In some embodiments, treating comprises at least one of the following: a reduction in the frequency of attacks, a reduction in the severity of attacks, a reduction in ischemic tissue injury, and a reduction in digital ulceration.

In some embodiments, treating comprises a reduction in the frequency and/or severity of attacks.

In some embodiments, preventing comprises at least one of the following: the prevention of attacks, the prevention of ischemic tissue injury, and the prevention of digital ulceration.

### DETAILED DESCRIPTION OF THE INVENTION

A recent clinical trial for selexipag, an oral prostacyclin receptor agonist, evaluated the attack frequency of Raynaud's phenomenon in individuals with systemic sclerosis. The primary efficacy endpoint in the trial was a reduction in the number of Raynaud's phenomenon attacks per week versus placebo. Selexipag was titrated to the highest tolerated dose in each individual. Despite a robust study design and a similar dosing scheme to that provided in the FDA approved label for pulmonary arterial hypertension (PAH), selexipag did not meet the primary efficacy endpoint in the Raynaud's study; Denton et al. Ann Rheum Dis 2016 Jun. 10; FR10265:530-1.

2-(((1r,4r)-4-(((4-chlorophenyl)(phenyl)carbamoyloxy)methyl)cyclohexyl)methoxy)acetic acid (Compound 1), also known as APD811 or ralinepag, is disclosed in U.S. Patent Publication No. 2011/0053958, which is incorporated by reference herein in its entirety. Like selexipag, ralinepag is an oral prostacyclin receptor agonist. However, as described herein, ralinepag is suitable for the treatment of Raynaud's.

As used in the present specification, the following words and phrases are generally intended to have the meanings as set forth below, except to the extent that the context in which they are used indicates otherwise.

Compound 1: As used herein, “Compound 1” refers to 2-(((1r,4r)-4-(((4-chlorophenyl)(phenyl)carbamoyloxy)methyl)cyclohexyl)methoxy)acetic acid, including crystalline forms thereof. As a non-limiting example, Compound 1 may be present in the crystalline form disclosed in WO2009117095 (incorporated by reference herein in its entirety), which may be characterized by one or more of the following ° 20 values for the peaks in the PXRD spectrum: 8.9, 10.8, 11.9, 15.2, 16.4, 16.8, 18.9, 20.3, 207 and 21.5, wherein the reported peaks can vary by about ±0.2° 2θ.

TREAT, TREATING, OR TREATMENT: As used herein the term “treat,” “treating” or “treatment” refers to the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder.

PREVENT, PREVENTING, OR PREVENTION: As used herein, the term “prevent,” “preventing” or “prevention” means prevention of the occurrence or onset of one or more symptoms associated with a particular disorder and does not necessarily mean the complete prevention of a disorder. For example, the term “prevent,” “preventing” and “prevention” refers to the administration of therapy on a prophylactic or preventative basis to an individual who may ultimately manifest at least one symptom of a disease or condition but who has not yet done so. Such individuals can be identified since risk factors that are known to correlate with the subsequent occurrence of the disease. Alternatively, prevention therapy can be administered without prior identification of a risk factor, as a prophylactic measure. Delaying the onset of the at least one symptom can also be considered prevention or prophylaxis.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting (even if the severity of each is less than when experienced alone).

ADVERSE EVENT: As used herein, an “adverse event” is an untoward medical occurrence that is associated with treatment with Compound 1 or a pharmaceutically acceptable salt, solvate or hydrate thereof. In one embodiment, an adverse event is selected from headache, nausea, vomiting, and jaw pain. In one embodiment, an adverse event is selected from headache, nausea, vomiting, jaw pain, flushing, abnormal pulse rate, abnormal QT interval, sitting systolic blood pressure >160 mmHg, sitting diastolic blood pressure >100 mmHg, systolic blood pressure <90 mmHg, or a combination of one more of the foregoing. In one embodiment, an adverse event is selected from abdominal pain, nosebleed, muscle aches, feeling of warmth, palpitations, dizziness, itching, diarrhoea, chest pressure, joint aches, prickling or tingling skin sensation, and lowering of blood pressure. In one embodiment, an adverse event is selected from chest pain, chest discomfort, and erythema.

OPTIMIZED DOSE: As used herein, an “optimized dose” refers a therapeutic dose optimized to the needs of a specific individual and is the highest dose of Compound 1, or the dose of a pharmaceutically acceptable salt, solvate, or hydrate thereof that is equivalent to the highest dose of Compound 1, that elicits the biological or medicinal response in the individual that is being sought and that can be tolerated by the individual, as determined by the individual, optionally in consultation with the individual's healthcare practitioner. The amount of Compound 1 in an optimized dose may vary between individuals. Further, the amount of Compound 1 may vary from time to time for a given individual.

UP-TITRATION/UP-TITRATING: As used herein, “up-titration of” or “up-titrating” a compound refers to increasing the amount of a compound until the individual does not tolerate the increased amount. Up-titration can be achieved in one or more dose increments, which may be the same or different. In some embodiments, a method described herein comprises prescribing and/or administering Compound 1 or a pharmaceutically acceptable salt, solvate or hydrate thereof in an amount equivalent to 0.01 mg or 0.02 mg of Compound 1 daily for about one week, followed by up-titration as disclosed herein until an optimized dose is administered. Administration of the optimized dose may then continue as long as necessary.

It is understood that when the phrase “pharmaceutically acceptable salts, solvates and hydrates” or the phrase “pharmaceutically acceptable salt, solvate or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound 1 that are salts, it embraces pharmaceutically acceptable solvates and/or hydrates of such salts.

It will be apparent to those skilled in the art that the dosage forms described herein may comprise, as the active component, either Compound 1 or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art. See, e.g., pp. 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like.

As used herein, the term “greater than” is used interchangeably with the symbol > and the term less than is used interchangeably with the symbol <. Likewise, the term greater than or equal to is interchangeably with the symbol ≥, and the term less than or equal to is interchangeably with the symbol ≤.

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all of the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions and methods are clearly within the scope of the invention(s), as described herein. For example, the dosage amounts of Compound 1 or pharmaceutically acceptable salts, solvates or hydrates thereof disclosed herein can be replaced with dosage amounts for other salt or crystalline forms, formulations, and dosage regimens that exhibit bioequivalence to the specified amount of Compound 1 or pharmaceutically acceptable salts, solvates or hydrates thereof, including forms with 80-125% of the AUC and/or Cₘₐₓ as measured by methods disclosed in the FDA's Guidance for Industry for Bioavailability and Bioequivalence (Guidance for Industry: Bioavailability and Bioequivalence Studies for Orally Administered Drug Products—General Considerations. U.S. Department of Health and Human Services, Food and Drug Administration, Center for Drug Evaluation and Research (CDER), March 2003, Revision 1, www.fda.gov/cder/guidance/index.htm). For example, the dosage amounts of Compound 1 disclosed herein can be replaced with dosage amounts for other salt or crystalline forms, formulations, or dosage regimens that exhibit bioequivalence to 0.6 mg of Compound 1.

It is appreciated that certain features of the invention(s), which are, for clarity, described in the context of separate embodiments, can also be provided in combination in a single embodiment. For example, a method that recites prescribing Compound 1 or a pharmaceutically acceptable salt, solvate or hydrate thereof and a separate method of the invention reciting administering Compound 1 or a pharmaceutically acceptable salt, solvate or hydrate thereof can be combined into a single method reciting prescribing and administering Compound 1 or a pharmaceutically acceptable salt, solvate or hydrate thereof.

In some embodiments, Raynaud's is Raynaud's syndrome. In some embodiments, Raynaud's is Raynaud's disease. In some embodiments, Raynaud's is Raynaud's phenomenon. In some embodiments, Raynaud's is primary Raynaud's syndrome. In some embodiments, Raynaud's is secondary Raynaud's syndrome. In some embodiments, Raynaud's is primary Raynaud's disease. In some embodiments, Raynaud's is secondary Raynaud's disease. In some embodiments, Raynaud's is primary Raynaud's phenomenon. In some embodiments, Raynaud's is secondary Raynaud's phenomenon. In some embodiments, Raynaud's is Raynaud's disease secondary to systemic sclerosis. In some embodiments, Raynaud's is Raynaud's disease secondary to another autoimmune disease. In some embodiments, Raynaud's is Raynaud's syndrome secondary to systemic sclerosis. In some embodiments, Raynaud's is Raynaud's syndrome secondary to another autoimmune disease. In some embodiments, Raynaud's is Raynaud's phenomenon secondary to an autoimmune disease. In some embodiments, Raynaud's is Raynaud's phenomenon secondary to systemic sclerosis.

In some embodiments, Raynaud's is active Raynaud's Phenomenon.

In some embodiments, Raynaud's is refractory primary Raynaud's Phenomenon.

In some embodiments, Raynaud's is refractory secondary Raynaud's Phenomenon.

In some embodiments, Raynaud's is idiopathic (primary) Raynaud's.

In some embodiments, Raynaud's is limited cutaneous scleroderma-associated Raynaud's.

In some embodiments, Raynaud's is diffuse cutaneous Scleroderma-associated Raynaud's.

In some embodiments, Raynaud's is mixed connective tissue disease-associated Raynaud's.

In some embodiments, Raynaud's is Raynaud's is secondary to an autoimmune disease.

In some embodiments, Raynaud's is Raynaud's is secondary to a connective tissue disease.

In some embodiments, an individual is assessed using a Raynaud's Condition Score (RCS). In some embodiments, the RCS is reduced by, or by about, 1, 2, 3, 4, 5, 6, 7, 8, or 9.

In some embodiments, an individual is assessed using a Raynaud Severity Visual Analog Score (VAS). In some embodiments, the VAS is a 100 millimeter (mm) line where 0 mm (left boundary) represents Raynaud's disease of low severity, and 100 mm (right boundary) represents extremely severe Raynaud's disease. In some embodiments, the individual makes a vertical mark on the VAS to indicate the severity of Raynaud's disease over the past two weeks. In some embodiments, the Raynaud's severity score is the distance from the left boundary to the vertical mark in mm.

In some embodiments, an individual is assessed using a Raynaud's Condition Score (RCS) Visual Analog Scale (VAS). In some embodiments, the VAS is a 100 millimeter (mm) line where 0 mm (left boundary) represents no difficulty with Raynaud's disease, and 100 mm (right boundary) represents extreme difficulty with Raynaud's disease. In some embodiments, the individual makes a vertical mark on the VAS to indicate difficulty experienced that day with Raynaud's disease. In some embodiments, the RCS is the distance from the left boundary to the vertical mark in mm.

In some embodiments, an individual has a VAS pain score of ≥25 mm of 100 mm.

In some embodiments, laser Doppler imaging of the hands is conducted.

In some embodiments, a cold exposure test is conducted. For example, in some embodiments, each digit temperature is measured and recorded at baseline, and then measured and re-recorded after 3-minute intervals following a 20 second 4 degree Celsius ice bath immersion.

In some embodiments, cold-induced vasospasm is evaluated.

In some embodiments, change in health assessment questionnaire (HAQ) is evaluated.

In some embodiments, change in digital ulcer number is evaluated.

In some embodiments, change in peak systolic flow is evaluated. For example, in some embodiments, change in digital artery flow velocity in the palmar digital artery is evaluated.

In some embodiments, time-averaged peak velocity blood flow is evaluated.

In some embodiments, the temperature difference between finger tips and the dorsum of the same hand is evaluated.

In some embodiments, quantitative changes in blood flow in the fingers of the non-dominant hand is measured after the clinical induction of constriction of blood vessels by exposure to local cold temperatures.

In some embodiments, quantitative reduction in skin temperature recovery time is evaluated. In some embodiments, symptoms of pain, tingling and/or numbness associated with Raynaud's phenomenon are evaluated. In some embodiments, and individual is evaluated using a patient hand symptom analog assessment score.

In some embodiments, the individual has rheumatoid arthritis. In some embodiments, the individual has systemic lupus erythematosus. In some embodiments, the individual has systemic sclerosis. In some embodiments, the diagnosis of systemic sclerosis is as defined by the European League Against Rheumatism (EULAR) criteria. In some embodiments, the individual has limited scleroderma (CREST). In some embodiments, the individual has Sjogren's syndrome. In some embodiments, the individual has primary Sjogren's syndrome. In some embodiments, the individual has mixed connective tissue disease.

In some embodiments, the individual is also administered an antiplatelet agent. In some embodiments, the individual is also administered a vasodilator. In some embodiments, the individual is also administered a calcium channel blocker. In some embodiments, the individual is also administered a PDE5 inhibitor. In some embodiments, the individual is also administered a phosphodiesterase inhibitor (e.g., sildenafil, tadalafil or vardenafil). In some embodiments, the individual is also administered an endothelin antagonist. In some embodiments, the individual is also administered an alpha-adrenergic antagonist. In some embodiments, the individual is also administered a pain medication.

In some embodiments, the methods of treatment provided herein are maintenance treatments for an individual who has previously received or is currently receiving another therapy for Raynaud's. In some embodiments, the individual with Raynaud's has been resistant to previous Raynaud's therapy. For example, in some embodiments, the individual with Raynaud's has been resistant to Ca2+ blocker, anti-platelet, topical nitroglycerin, ACE inhibitor or ARB, alpha blocker, SSRI or other anti-anxiolytic, PDE5, ETRA, or IV prostacyclin analogue therapy. In some embodiments, the individual with Raynaud's has been resistant to vasodilatory therapy. In some embodiments, the individual with Raynaud's has been resistant to intravenous prostanoid therapy. In some embodiments, the individual with Raynaud's has been resistant to epoprostenol, iloprost, bosentan, tadalafil, nifedipine, nicardipine, or quinapril therapy.

In some embodiments, the Compound 1, or a pharmaceutically acceptable salt, hydrate, or solvate thereof, is administered orally.

In some embodiments, the Compound 1, or a pharmaceutically acceptable salt, hydrate, or solvate thereof, is formulated as a capsule suitable for oral administration. In some embodiments, the Compound 1, or a pharmaceutically acceptable salt, hydrate, or solvate thereof, is formulated as a tablet suitable for oral administration.

In some embodiments, the Compound 1, or a pharmaceutically acceptable salt, hydrate, or solvate thereof, is Compound 1, or a hydrate or solvate thereof.

In some embodiments, the Compound 1, or a pharmaceutically acceptable salt, hydrate, or solvate thereof, is Compound 1.

In some embodiments, the dose of Compound 1 is selected from, or from about, 0.01 mg, 0.02 mg, 0.025 mg, 0.03 mg, 0.04 mg, 0.05 mg, 0.06 mg, 0.065 mg, 0.07 mg, 0.075 mg, 0.08 mg, 0.09 mg, 0.1 mg, 0.12 mg, 0.15 mg, 0.16 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, and 1.5 mg daily. In some embodiments, the dose of Compound 1 is from 0.4 to 1.0 mg daily. In some embodiments, the dose of Compound 1 is from 0.6 to 1.0 mg daily. In some embodiments, the dose of Compound 1 is from 0.6 to 0.8 mg daily. In some embodiments, the dose of Compound 1 is from 0.65 to 1.0 mg daily. In some embodiments, the dose of Compound 1 is from 0.65 to 0.8 mg daily. In some embodiments, the dose of Compound 1 is determined by individual tolerability. In some embodiments, the dose of Compound 1 is greater than 0.4 mg daily. In some embodiments, the dose of Compound 1 is greater than 0.6 mg daily. In some embodiments, the dose of Compound 1 is greater than 0.8 mg daily. In some embodiments, the daily dose is administered once per day. In some embodiments, the daily dose is administered twice per day. In some embodiments, the dose is an optimized dose. In some embodiments, the dose is a maintenance dose. In some embodiments, the maintenance dose is the maximum tolerated dose for the individual.

In some embodiments, the amount of Compound 1 administered to the individual is selected from, or from about: 0.01 mg, 0.15 mg, 0.02 mg, 0.025 mg, 0.03 mg, 0.35 mg, 0.04 mg, 0.45 mg, 0.05 mg, 0.55 mg, 0.06 mg, 0.065 mg, 0.07 mg, 0.075 mg, 0.08 mg, 0.85 mg, 0.09 mg, 0.95 mg, 0.1 mg, 0.11 mg, 0.12 mg, 0.13 mg, 0.14 mg, 0.15 mg, 0.16 mg, 0.17 mg, 0.175 mg, 0.18 mg, 0.19 mg, 0.2 mg, 0.225 mg, 0.25 mg, 0.275 mg, 0.3 mg, 0.325 mg, 0.35 mg, 0.375 mg, 0.4 mg, 0.425 mg, 0.45 mg, 0.475 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, and 1.5 mg.

In some embodiments, the amount of Compound 1 administered to the individual is selected from, or from about: 0.01 mg, 0.15 mg, 0.02 mg, 0.025 mg, 0.03 mg, 0.35 mg, 0.04 mg, 0.45 mg, 0.05 mg, 0.55 mg, 0.06 mg, 0.065 mg, 0.07 mg, 0.075 mg, 0.08 mg, 0.85 mg, 0.09 mg, 0.95 mg, 0.1 mg, 0.11 mg, 0.12 mg, 0.13 mg, 0.14 mg, 0.15 mg, 0.16 mg, 0.17 mg, 0.175 mg, 0.18 mg, 0.19 mg, 0.2 mg, 0.225 mg, 0.25 mg, 0.275 mg, 0.3 mg, 0.325 mg, 0.35 mg, 0.375 mg, 0.4 mg, 0.425 mg, 0.45 mg, 0.475 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, and 1.5 mg daily.

In some embodiments, the methods or uses provided herein involve a pharmaceutical composition comprising an amount of Compound 1, or a pharmaceutically acceptable salt, hydrate, or solvate thereof, equivalent to, equivalent to about, or equivalent to at least about, 0.01 mg, 0.02 mg, 0.025 mg, 0.03 mg, 0.04 mg, 0.05 mg, 0.06 mg, 0.065 mg, 0.07 mg, 0.075 mg, 0.08 mg, 0.09 mg, 0.1 mg, 0.12 mg, 0.15 mg, 0.16 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, or 1.5 mg of Compound 1.

In some embodiments, the methods or uses provided herein involve a pharmaceutical composition comprising an amount of Compound 1, or a pharmaceutically acceptable salt, hydrate, or solvate thereof, equivalent to, or equivalent to about, 0.01 mg to 1.0 mg of Compound 1, such as 0.01 mg to 0.6 mg of Compound 1, such as 0.01 mg to 0.3 mg of Compound 1, such as 0.01 mg to 0.1 mg of Compound 1, such as 0.02 mg to 0.08 mg of Compound 1, such as 0.03 mg to 0.06 mg of Compound 1, or such as 0.04 mg of Compound 1.

In some embodiments involving a pharmaceutical composition, the composition is in the form of a capsule or tablet. In some embodiments of the pharmaceutical composition, the composition is in the form of a capsule. In some embodiments of the pharmaceutical composition, the composition is in the form of a tablet.

The compounds disclosed herein are useful in the treatment of Raynaud's and symptoms thereof. Symptoms of Raynaud's syndrome include, for example, digital ulceration and gangrene. The compounds disclosed herein are useful in the treatment of symptoms of Raynaud's syndrome.

Also provided are methods for the treatment of Raynaud's in an individual in need thereof, comprising prescribing and/or administering to the individual Compound 1 or a pharmaceutically acceptable salt, hydrate or solvate thereof, or a pharmaceutical composition thereof, via a titration scheme as disclosed herein. Also provided are methods for the treatment of Raynaud's in an individual in need thereof, comprising administering to the individual Compound 1 via a titration scheme as disclosed herein. In some embodiments, Compound 1 is titrated over, or over about, 1, 2, 3, 4, 5, 6, 7, 8, 9, 10, 11, 12, 13, 14, 15, 16, 17, 18, 19, or 20 weeks. In some embodiments, Compound 1 is titrated over, or over about, 9 weeks. In some embodiments, Compound 1 is titrated over, or over about, 16 weeks. In some embodiments, Compound 1 is titrated in increments of 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 mg. In some embodiments, Compound 1 is titrated in increments of 0.5 mg. In some embodiments, Compound 1 is titrated to a maximum tolerated dose in the individual.

In some embodiments of the methods for the treatment of Raynaud syndrome, the Compound 1 or pharmaceutically acceptable salt, hydrate, or solvate thereof is formulated as a capsule or tablet suitable for oral administration. In some embodiments, the Compound 1, or a pharmaceutically acceptable salt, hydrate, or solvate thereof, is formulated as a capsule. In some embodiments, the Compound or pharmaceutically acceptable salt, hydrate, or solvate thereof is formulated as a tablet.

In some embodiments, the treatment or prevention of Raynaud's is determined by a change in Raynaud's Condition Score, number of Raynaud's attacks, duration of Raynaud's attacks, reduction in ulcer burden in secondary Raynaud's, Raynaud Severity Visual Analog Score (VAS), Raynaud's Condition Score (RCS) Visual Analog Score (VAS), digital blood pressure, or capillary diameter.

Also provided herein is the use of Compound 1, or a pharmaceutically acceptable salt, hydrate, or solvate thereof, as described herein, in the manufacture of a medicament for the treatment of Raynaud's as described herein.

Also provided herein is Compound 1, or a pharmaceutically acceptable salt, hydrate, or solvate thereof, as described herein, for use in a method of treatment of Raynaud's as described herein.

Also provided herein is Compound 1, or a pharmaceutically acceptable salt, hydrate, or solvate thereof, as described herein, for the treatment of Raynaud's as described herein.

Also provided herein is a kit comprising a titration package as disclosed herein and instructions indicating that the medication is to be administered to an individual in need of treatment for Raynaud's.

Also provided herein is a method of treating Raynaud's comprising providing a titration package as disclosed herein to an individual in need thereof.

In some embodiments, Compound 1 or a pharmaceutically acceptable salt, solvate or hydrate thereof is administered in a tablet or a capsule suitable for oral administration. In some embodiments, Compound 1 or a pharmaceutically acceptable salt, solvate or hydrate thereof is administered in a capsule suitable for oral administration.

Conventional excipients, such as binding agents, fillers, acceptable wetting agents, tabletting lubricants and disintegrants can be used in tablets and capsules for oral administration. Liquid preparations for oral administration can be in the form of solutions, emulsions, aqueous or oily suspensions and syrups. Alternatively, oral preparations can be in the form of dry powder that can be reconstituted with water or another suitable liquid vehicle before use. Additional additives such as suspending or emulsifying agents, non-aqueous vehicles (including edible oils), preservatives and flavorings and colorants can be added to the liquid preparations.

Parenteral dosage forms can be prepared by dissolving the compound in a suitable liquid vehicle and filter sterilizing the solution before filling and sealing an appropriate vial or ampule. These are just a few examples of the many appropriate methods well known in the art for preparing dosage forms. Suitable pharmaceutically-acceptable carriers, outside those mentioned herein, are known in the art. See, e.g., Remington, The Science and Practice of Pharmacy, 20^{th} Edition, 2000, Lippincott Williams & Wilkins, (Editors: Gennaro et al.).

The compounds provided herein can be administrated in a wide variety of oral and parenteral dosage forms. It will be obvious to those skilled in the art that the dosage forms may comprise, as the active component, either a compound provided herein or a pharmaceutically acceptable salt, solvate or hydrate of a compound provided herein.

In some embodiments, Compound 1 is in a pharmaceutical formulation or further comprises a pharmaceutically acceptable carrier. Pharmaceutical formulations include those suitable for oral, rectal, nasal, topical (including buccal and sub-lingual), vaginal or parenteral (including intramuscular, subcutaneous and intravenous) administration or in a form suitable for administration by inhalation, insufflation, or by transdermal patch. The compounds provided herein, together with a conventional adjuvant, carrier, or diluent, can thus be placed into the form of pharmaceutical formulations and unit dosages thereof and in such form may be employed as solids, such as tablets or filled capsules, or liquids, such as solutions, suspensions, emulsions, elixirs, gels or capsules filled with the same, all for oral use, in the form of suppositories for rectal administration, or in the form of sterile injectable solutions for parenteral (including subcutaneous) use. The active ingredient may also be administered by injection as a composition wherein, for example, saline, dextrose or water may be used as a suitable pharmaceutically acceptable carrier. Such pharmaceutical compositions and unit dosage forms thereof can comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition can be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate.

Some embodiments include a method of producing a pharmaceutical composition for “combination therapy,” comprising admixing at least one compound according to any of the compound embodiments disclosed herein, together with at least one known pharmaceutical agent as described herein and a pharmaceutically acceptable carrier.

Further embodiments include the embodiment disclosed in the following Examples, which are not to be construed as limiting in any way.

### EXAMPLES

### Example 1—Clinical Trial 1

A 22-week randomized, double-blind, placebo-controlled study with a dose titration period of up to 9 weeks was conducted. Sixty-one individuals were randomized 2:1 Compound 1 to placebo. Right Heart Catheterization (RHC) measurements were obtained prior to study Day 1 of the dose titration period and at Week 22. The following values were obtained and recorded: pulmonary artery pressure (PAP) (systolic, diastolic, and mean), heart rate (HR), right atrial pressure (RAP), pulmonary capillary wedge pressure (PCWP) right ventricular pressure (RVP) and cardiac output (CO), pulmonary vascular resistance (PVR), arterial and mixed venous oxygen saturation (FiO2) (if applicable). Systemic vascular resistance (SVR) was estimated from blood pressure measurements.

Primary efficacy endpoints for the study were: a) change from baseline in PVR after 22 weeks of treatment, and b) change from baseline in 6 MWD after 22 weeks of treatment. Compound 1 was administered as a capsule in 0.01, 0.02, 0.03, 0.04, and 0.10 mg dose strengths. The starting dose of Compound 1 was 0.01 mg twice daily. The dose of Compound 1 was titrated according to individual tolerability. If the initial dose was tolerated (0.01 mg twice daily), then the dose was increased once a week in the following fashion: 0.02 mg twice daily, 0.03 mg twice daily, 0.04 mg twice daily, 0.06 mg twice daily, 0.08 mg, 0.1 mg twice daily, 0.2 mg twice daily and 0.3 mg twice daily. The dose was optionally escalated to a possible maximum total daily dose of 0.6 mg (0.3 mg twice daily), pending tolerability. If a dose was not tolerated, Compound 1 was optionally decreased to the previous dose level. If the initial dose of 0.01 mg twice daily was not tolerated, dosing was optionally decreased to 0.01 mg once daily.

Compound 1 achieved the primary endpoint with a statistically significant change from baseline in pulmonary vascular resistance (PVR) compared to placebo. Compound 1 also demonstrated numerical improvement in 6-minute walk distance (6MWD). Adverse events observed in the study were consistent with other prostacyclin treatments for the management of PAH. The distribution of maintenance doses for individuals receiving Compound 1 was as follows: 0.02 mg (n=1), 0.03 mg (n=1), 0.04 mg (n=0), 0.06 mg (n=3), 0.08 mg (n=3), 0.12 mg (n=5), 0.16 mg (n=4), 0.2 mg (n=6), 0.4 mg (n=12), and 0.6 mg (n=5).

### Example 2—Clinical Trial 2

A randomized, double-blind, placebo-controlled study is conducted in individuals with severe Raynaud's. Individuals are administered Compound 1 using a titration scheme. Compound 1 is determined to be efficacious for the treatment and/or prevention of Raynaud's.

### Example 3—Clinical Trial 3

Individuals with Raynaud's phenomenon will be randomized to receive Compound 1 or placebo in a crossover study. Individuals will keep a daily record of the number of Raynaud's phenomenon attacks they experience per day and the duration of each attack. Individuals may be assessed using a Raynaud Severity Visual Analog Score (VAS) and/or a Raynaud's Condition Score (RCS) Visual Analog Scale (VAS). Individuals will also be evaluated for digital blood pressure and/or capillary diameter. The frequency and severity of adverse events will be measured. Data will be collected at multiple visits during the study.

### Example 4—Clinical Trial 4

Digital blood flow will be measured during a cold exposure test in individuals suffering from Raynaud's phenomenon. Measurements will be compared a.) in a first time period in which an individual will be administered Compound 1 and b.) in a second time period in which the same individual will be administered placebo. Individuals will be randomized for the order in which the first and second time periods occur. Individuals will be evaluated for blood pressure, pulse rate, changes in clinical laboratory and hematology assessments, changes in digital blood flow at room temperature (e.g., as measured by laser Doppler perfusion imaging), and/or changes in digital blood flow during cold exposure (e.g., as measured by laser Doppler perfusion imaging). The frequency and severity of adverse events will be measured.

### Example 5—Preparation of Compound 1

The preparation of Compound 1 is described in International Patent Application No. PCT/US2009/001688 (referred to as Compound 22 therein), published as International Publication No. WO2009/117095, the entire contents of which are incorporated herein by reference in their entirety. Representative processes include, Examples 1.22 and 1.115 for the preparation of 2-(((1r,4r)-4-(((4-chlorophenyl)(phenyl)carbamoyloxy)methyl)cyclohexyl)methoxy)acetic acid; Example 1.106 for the preparation of sodium 2-(((1r,4r)-4-(((4-chlorophenyl)(phenyl)carbamoyloxy)methyl)cyclohexyl) methoxy)acetate; and Example 1.107 for the preparation of sodium 2-(((1r,4r)-4-(((4-chlorophenyl) (phenyl)carbamoyloxy)methyl)cyclohexyl)methoxy)acetate hydrate. Certain crystal forms of Compound 1 are also disclosed.

Other uses of the disclosed methods will become apparent to those in the art based upon, inter alia, a review of this patent document.

What is claimed is:

1. A method of treating or preventing Raynaud's, comprising:
prescribing and/or administering to an individual in need thereof 2-(((1r,4r)-4-(((4-chlorophenyl)(phenyl)carbamoyloxy)methyl)cyclohexyl)methoxy)acetic acid (Compound 1) or a pharmaceutically acceptable salt, hydrate, or solvate thereof.

2. The method of claim 1, wherein the individual has primary Raynaud's.

3. The method of claim 1, wherein the individual has secondary Raynaud's.

4. The method of claim 1, wherein the individual has at least one digital ulcer.

5. The method of claim 1, wherein the individual has digital gangrene.

6. The method of claim 1, wherein the individual has systemic sclerosis.

7. The method of claim 1, wherein the individual has Raynaud's secondary to a condition selected from: systemic sclerosis, rheumatoid arthritis, atherosclerosis, cryoglobulinemia, hypothyroidism, injury, and drug reaction.

8. The method of claim 1, wherein the individual has Raynaud's secondary to systemic sclerosis.

9. The method of claim 1, wherein the individual has at least one digital ulcer and systemic sclerosis.

10. The method of any one of the preceding claims, wherein the individual is administered an amount equivalent to 0.01 mg to 1.5 mg of Compound 1 daily.

11. The method of any one of claims 1 to 9, wherein the individual is administered an amount equivalent to up to 0.6 mg of Compound 1 daily.

12. The method of any one of claims 1 to 9, wherein the dose of the Compound 1 or pharmaceutically acceptable salt, hydrate, or solvate thereof is selected from: 0.01 mg, 0.02 mg, 0.025 mg, 0.03 mg, 0.04 mg, 0.05 mg, 0.06 mg, 0.065 mg, 0.07 mg, 0.075 mg, 0.08 mg, 0.09 mg, 0.1 mg, 0.12 mg, 0.15 mg, 0.16 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, and 1.5 mg daily.

13. The method of any one of the preceding claims, wherein the dose is administered once daily.

14. The method of any one of claims 1 to 12, wherein the dose is administered twice daily.

15. The method of any one of claims 1 to 12, wherein the Compound 1 or pharmaceutically acceptable salt, hydrate, or solvate thereof is suitable for administration once daily.

16. The method of any one of claims 1 to 12, wherein the Compound 1 or pharmaceutically acceptable salt, hydrate, or solvate thereof is suitable for administration twice daily.

17. The method of any one of the preceding claims, wherein the Compound 1 or pharmaceutically acceptable salt, hydrate, or solvate thereof is administered via a titration scheme that comprises up-titration of the Compound 1 or pharmaceutically acceptable salt, hydrate, or solvate thereof.

18. The method of claim 17, wherein the titration scheme comprises increasing the dose, wherein the cycle is repeated so long as the individual tolerates a further increased dose, until an optimized dose is administered.

19. The method of claim 18, wherein the optimized dose is selected from: 0.01 mg, 0.02 mg, 0.025 mg, 0.03 mg, 0.04 mg, 0.05 mg, 0.06 mg, 0.065 mg, 0.07 mg, 0.075 mg, 0.08 mg, 0.09 mg, 0.1 mg, 0.12 mg, 0.15 mg, 0.16 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, and 1.5 mg daily.

20. The method of claim 19, wherein the optimized dose is administered once daily.

21. The method of claim 19, wherein the optimized dose is administered twice daily.

22. The method of any one of the preceding claims, wherein the Compound 1 or pharmaceutically acceptable salt, hydrate, or solvate thereof is administered orally.

23. The method of any one of the preceding claims, wherein the Compound 1 or pharmaceutically acceptable salt, hydrate, or solvate thereof is in the form of a tablet.

24. The method of any one of the preceding claims, wherein the Compound 1 or pharmaceutically acceptable salt, hydrate, or solvate thereof is in the form of a capsule.

25. The method of any one of the preceding claims, wherein the Compound 1, or a pharmaceutically acceptable salt, hydrate, or solvate thereof, is Compound 1.

26. The method of any one of the preceding claims, wherein treating comprises at least one of the following: a reduction in the frequency of attacks, a reduction in the severity of attacks, a reduction in ischemic tissue injury, and a reduction in digital ulceration.

27. The method of any one of the preceding claims, wherein treating comprises a reduction in the frequency and/or severity of attacks.

28. The method of any one of the preceding claims, wherein preventing comprises at least one of the following: the prevention of attacks, the prevention of ischemic tissue injury, and the prevention of digital ulceration.
